(19) United States
(12) Patent Application Publication
(10) Pub. No.: US 2025/0152473 A1
(43) Pub. Date: May 15, 2025

(54) STABLE PLINABULIN FORMULATIONS AND METHODS OF THEIR PREPARATION AND USE

(71) Applicant: BeyondSpring Pharmaceuticals, Inc., Florham Park, NJ (US)

(72) Inventors: Lan Huang, Florham Park, NJ (US); George Kenneth Lloyd, Florham Park, NJ (US); Jianmin Mao, Florham Park, NJ (US); Christopher G. Boiko, Florham Park, NJ (US); Hao Zhang, Florham, NJ (US)

(21) Appl. No.: 18/837,546

(22) PCT Filed: Feb. 8, 2023
(86) PCT No.: PCT/US2023/062222

§ 371 Date: Aug. 9, 2024

Related U.S. Application Data
(60) Provisional application No. 63/337,256, filed on May 2, 2022.
(60) Provisional application No. 63/308,723, filed on Feb. 10, 2022.

(51) Int. Cl. A61J 1/14; A61K 31/496; A61K 47/10

(52) U.S. Cl. CPC A61J 1/1468; A61J 1/1412; A61K 31/496; A61K 47/10

(57) ABSTRACT

Described herein are plinabulin unit dose forms, kits, and methods of preparing and using plinabulin. Some embodiments relate to the use of light-protective containers, light-protective packaging, light-protected IV bags, and light-protected IV tubing.

### CROSS REFERENCE TO RELATED APPLICATIONS

This patent application claims the benefit of priority to U.S. Provisional Application No. 63/308,723, filed Feb. 10, 2022, and U.S. Provisional Application No. 63/337,256, filed May 2, 2022. All of the foregoing applications are fully incorporated herein by reference in their entireties for all purposes.

### FIELD OF THE INVENTION

The present disclosure relates to the field of chemistry and medicine. More particularly, the present disclosure relates to stable plinabulin formulations and methods of their preparation and use.

### BACKGROUND

Many formulations comprising therapeutic agents are sensitive to one or more elements or conditions, which render them unstable after some time. One such element or condition is sensitivity to one or more elements of the air or light. Another such element or condition is the presence of gases or dissolved gases in the formulation, or exposure of one or more elements of the formulation to light, contaminating materials or organisms such as bacteria. Another such condition or element is the amount of a particular molecule relative to the amount of one or more components in a formulation, including but not limited to the active agent of the formulation. Thus, there is a need for formulations, including pharmaceutical formulations, that retain the stability of one or more of a therapeutic agent or other elements present in a formulation comprising a therapeutic agent when the formulation is stored for a period of time or for the time the therapeutic agent is administered.

### SUMMARY OF THE INVENTION

The present disclosure provides stable plinabulin formulations and methods of their preparation and use. In aspects, the disclosure describes a vessel. In some embodiments, the vessel includes a liquid formulation comprising plinabulin, propylene glycol, and polyoxyl 15 hydroxystearates (PEG15-hydroxystearates), wherein the vessel is configured to block at least 85% of UV/visible light between 290 nm and 450 nm. In some embodiments, the vessel is a single use amber glass vial. In some embodiments, the amber glass vial further comprises a rubber stopper and a flip-off cap. In some embodiments, the rubber stopper is a chlorobutyl rubber stopper with a B2-40 coating. In some embodiments, the rubber stopper further comprises a plug portion, wherein the plug portion comprises a Flurotec coating. In some embodiments, the vessel is an IV bag. In some embodiments, the vessel is an IV bag covered by a light-protective sleeve. In some embodiments, the vessel is IV tubing within a light-protective sleeve. In some embodiments, the plinabulin is at a concentration of about 40 mg/10 ml in the liquid formulation. In some embodiments, the liquid formulation includes 60:40 (wt:wt) of propylene glycol and polyoxyl 15 hydroxystearates (PEG15-hydroxystearates). In some embodiments, the liquid formulation comprises less than 0.5% ether impurity. In some embodiments, the liquid formulation comprises less than 0.5% alcohol impurity. In some embodiments, the liquid formulation comprises less than 0.5% water. In some embodiments, the liquid formulation comprises less than 2.0% total impurities.

In aspects, the disclosure describes a kit. In some embodiments, the kit includes plinabulin in a light-protective container and a light-protective packaging, wherein, the light-protective container blocks at least 85% of UV/visible light between 290 nm and 450 nm. In some embodiments, the plinabulin is at a concentration of about 40 mg/10 ml in a liquid formulation. In some embodiments, the plinabulin is in a liquid formulation comprising propylene glycol and polyoxyl 15 hydroxystearates (PEG15-hydroxystearates). In some embodiments, the liquid formulation comprises 60:40 (wt:wt) of propylene glycol and polyoxyl 15 hydroxystearates (PEG15-hydroxystearates). In some embodiments, the light-protective container is an amber vial. In some embodiments, the light-protective container further comprises a rubber stopper and a flip-off cap. In some embodiments, the rubber stopper is a chlorobutyl rubber stopper with a B2-40 coating. In some embodiments, the rubber stopper further comprises a plug portion, wherein the plug portion comprises a Flurotec coating. In some embodiments, the light-protective packaging comprises solid black printing on its interior. In some embodiments, the kit further includes an IV bag. In some embodiments, the IV bag is configured to block at least 85% of UV/visible light between 290 nm and 450 nm. In some embodiments, the IV bag comprises a light-protective sleeve. In some embodiments, the kit further includes IV tubing. In some embodiments, the IV tubing is configured to block at least 85% of UV/visible light between 290 nm and 450 nm. In some embodiments, the IV tubing comprises a light-protective sleeve.

In aspects, the disclosure describes a method for administering plinabulin to a subject. In some embodiments, the method of administering plinabulin to a subject in need thereof includes providing plinabulin in a light-protected IV bag and administering plinabulin to a subject in need thereof through light-protected IV tubing. In some embodiments, the light-protected IV bag blocks at least 85% of UV/visible light between 290 nm and 450 nm. In some embodiments, the light-protected IV tubing blocks at least 85% of UV/visible light between 290 nm and 450 nm. In some embodiments, the method includes providing the plinabulin comprises injecting the plinabulin into the light-protected IV bag. In some embodiments, the method includes providing the plinabulin comprises injecting the plinabulin into a non-light-protected IV bag and then placing a light-protecting sleeve over the non-light-protected IV bag. In some embodiments, the plinabulin is injected as a liquid formulation into D5W. In some embodiments, the liquid formulation comprises propylene glycol, and polyoxyl 15 hydroxystearates (PEG15-hydroxystearates). In some embodiments, the liquid formulation comprises 60:40 (wt:wt) of propylene glycol and polyoxyl 15 hydroxystearates (PEG15-hydroxystearates). In some embodiments, the liquid formulation comprises plinabulin is at a concentration of about 40 mg/10 ml. In some embodiments, the method includes placing non-light-protected IV tubing within a light-protected sleeve to form the light-protected IV tubing.

### BRIEF DESCRIPTION OF THE DRAWINGS

FIG. 1 is an illustration of a box of an embodiment of the disclosure.

FIG. 2 is a picture of an amber IV bag cover of an embodiment of the disclosure.

FIG. 3 is a picture of an amber IV tubing cover of an embodiment of the disclosure.

### DETAILED DESCRIPTION

Described herein are plinabulin unit dose forms, kits, and methods of preparing and using plinabulin compositions. Plinabulin, (3Z,6Z)-3-Benzylidene-6-{[5-(2-methyl-2-propanyl)-1H-imidazol-4-yl]methylene}-2,5-piperazinedione, is a synthetic analog of the natural compound phenylahistin. Plinabulin can be readily prepared according to methods and procedures detailed in U.S. Pat. Nos. 7,064,201 and 7,919,497, which are incorporated herein by reference in their entireties.

Before the present disclosure is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

The term “agent” is used herein to denote a chemical compound, a mixture of chemical compounds, a biological macromolecule, or an extract made from biological materials.

The terms “cancer”, “neoplasm”, and “carcinoma”, are used interchangeably herein to refer to cells which exhibit relatively autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. Detection of cancerous cells is of particular interest.

The term “subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The terms “effective amount” or a “therapeutically effective amount” as used herein refers to an amount of a therapeutic agent that is effective to relieve, to some extent, or to reduce the likelihood of onset of, one or more of the symptoms of a disease or condition, and can include curing a disease or condition.

The term “liquid formulation,” as used herein refers to pharmaceutical formulations which contain a mixture of active drug components and nondrug components (excipients) dissolved or suspended in a suitable solvent or mixtures of solvents. Liquid dosage forms may be supplied as ready-to-use liquids or powders for reconstitution. Liquid dosage forms may be in monophasic liquid dosage forms and in biphasic liquid dosage forms.

It is generally preferred to administer the compounds of the disclosure in an intravenous or subcutaneous unit dosage form; however, other routes of administration are also contemplated. Contemplated routes of administration include but are not limited to oral, parenteral, intravenous, and subcutaneous. The compounds of preferred embodiments can be formulated into liquid preparations for, e.g., oral administration. Suitable forms include suspensions, syrups, elixirs, and the like. Oral liquids may typically be nonsterile, whereas liquids administered by parenteral routes may be available as sterile and nonsterile formulations.

The compounds of the disclosure can be in an admixture with a suitable carrier, diluent, or excipient such as sterile water, physiological saline, glucose, or the like, and can contain auxiliary substances such as wetting or emulsifying agents, pH buffering agents, gelling or viscosity enhancing additives, preservatives, flavoring agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Ansel, H. (2010). Pharmaceutical Calculations, (13th ed.). London: Wolters Kluwer Health and Lippincott Williams & Wilkins. Chaudhari, S. and Patil, P. (2012). Pharmaceutical Excipients: A review. International Journal of Advances in Pharmacy, Biology and Chemistry, 1(1): 21-34. Denton, P. and Rostron, C (2013). Pharmaceutics: The Science of Medicine Design. United Kingdom: Oxford University Press. Gautami, J. (2016). Liquid Dosage Forms. Nano Science & Nano Technology, 10(3):1-9.

Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components may influence the physical state, solubility, stability, rate of in vivo release, and rate of in vivo clearance, and are thus chosen according to the intended application, such that the characteristics of the carrier are tailored to the selected route of administration.

When administered orally in liquid form, a liquid carrier such as water, petroleum, oils of animal or plant origin such as peanut oil, mineral oil, soybean oil, or sesame oil, or synthetic oils can be added to the active ingredient(s). Physiological saline solution, dextrose, or other saccharide solution, or glycols such as ethylene glycol, propylene glycol, or polyethylene glycol are also suitable liquid carriers. The pharmaceutical compositions can also be in the form of oil-in-water emulsions. The oily phase can be a vegetable oil, such as olive or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions can also contain sweetening and flavoring agents.

When a compound of the preferred embodiments is administered by intravenous, parenteral, or other injection, it is preferably in the form of a pyrogen-free, parenterally acceptable aqueous solution or oleaginous suspension. Suspensions can be formulated according to methods well known in the art using suitable dispersing or wetting agents and suspending agents. The preparation of acceptable aqueous solutions with suitable pH, isotonicity, stability, and the like, is within the skill in the art. A preferred pharmaceutical composition for injection preferably contains an isotonic vehicle such as 1,3-butanediol, water, isotonic sodium chloride solution, Ringer's solution, dextrose solution, dextrose and sodium chloride solution, lactated Ringer's solution, or other vehicles as are known in the art. In addition, sterile fixed oils can be employed conventionally as a solvent or suspending medium. For this purpose, any bland fixed oil can be employed including synthetic mono or diglycerides. In addition, fatty acids such as oleic acid can likewise be used in the formation of injectable preparations. The pharmaceutical compositions can also contain stabilizers, preservatives, buffers, antioxidants, or other additives known to those of skill in the art.

The duration of an injection can be adjusted depending upon various factors, and can comprise a single injection administered over the course of a few seconds or less, to 0.5, 0.1, 0.25, 0.5, 0.75, 1, 2, 3, 4, 5, 6, 7, 8, 9, 10, 11, 12, 13, 14, 15, 16, 17, 18, 19, 20, 21, 22, 23, or 24 hours or more of continuous intravenous administration.

The terms “treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

### Overview

Embodiments of the present disclosure may include a vessel comprising a liquid formulation including plinabulin, propylene glycol, and polyoxyl 15 hydroxystearates (PEG15-hydroxystearates). In some embodiments, the vessel may be configured to block at least 85% of UV/visible light between 290 nm and 450 nm. In some embodiments, the vessel may be a single use amber glass vial.

In some embodiments, the amber glass vial may include a rubber stopper and a flip-off cap. In some embodiments, the rubber stopper may be a chlorobutyl rubber stopper with a B2-40 coating. In some embodiments, the rubber stopper may include a plug portion. In some embodiments, the plug portion may include a Flurotec coating. In any of these preceding embodiments, the vessel may comprise a liquid formulation including plinabulin, propylene glycol, and polyoxyl 15 hydroxystearates (PEG15-hydroxystearates). In any of these preceding embodiments, the vessel may comprise a liquid formulation including plinabulin and any other suitable liquid formulation.

In some embodiments, the vessel may be an IV bag covered by a light-protective sleeve. In some embodiments, the vessel may be an IV bag. In some embodiments, the vessel may be IV tubing within a light-protective sleeve. In some embodiments, the plinabulin may be at a concentration of about 40 mg/10 ml in the liquid formulation. In any of these preceding embodiments, the vessel may comprise a liquid formulation including plinabulin, propylene glycol, and polyoxyl 15 hydroxystearates (PEG15-hydroxystearates). In any of these preceding embodiments, the vessel may comprise a liquid formulation including plinabulin and any other suitable liquid formulation.

In some embodiments, the vessel of any of the preceding embodiments may comprise 60:40 (wt:wt) of propylene glycol and polyoxyl 15 hydroxystearates (PEG15-hydroxystearates). In some embodiments, the vessel of any preceding embodiment may comprise a liquid formulation, the liquid formulation may include less than 0.5% ether impurity. In any of these preceding embodiments, the vessel may comprise a liquid formulation including plinabulin, propylene glycol, and polyoxyl 15 hydroxystearates (PEG15-hydroxystearates). In any of these preceding embodiments, the vessel may comprise a liquid formulation including plinabulin and any other suitable liquid formulation.

In some embodiments, the liquid formulation may include less than 0.5% alcohol impurity. In some embodiments, the liquid formulation may include less than 0.5% water. In some embodiments, the liquid formulation may include less than 2.0% total impurities. In any of these preceding embodiments, the vessel may comprise a liquid formulation including plinabulin, propylene glycol, and polyoxyl 15 hydroxystearates (PEG15-hydroxystearates). In any of these preceding embodiments, the vessel may comprise a liquid formulation including plinabulin and any other suitable liquid formulation.

Embodiments of the present disclosure may also include a kit, including plinabulin in a light-protective container. Embodiments may also include a light-protective packaging. In some embodiments, the light-protective container blocks at least 85% of UV/visible light between 290 nm and 450 nm. In some embodiments, the plinabulin may be at a concentration of about 40 mg/10 ml in a liquid formulation. In any of these preceding kit embodiments, the container may comprise a liquid formulation including plinabulin, propylene glycol, and polyoxyl 15 hydroxystearates (PEG15-hydroxystearates). In any of these preceding embodiments, the container may comprise a liquid formulation including plinabulin and any other suitable liquid formulation.

In some embodiments, the plinabulin may be in a liquid formulation including propylene glycol and polyoxyl 15 hydroxystearates (PEG15-hydroxystearates). In some embodiments, the liquid formulation may include 60:40 (wt:wt) of propylene glycol and polyoxyl 15 hydroxystearates (PEG15-hydroxystearates). In some embodiments, the light-protective container may be an amber vial.

In some embodiments, the light-protective container may include a rubber stopper and a flip-off cap. In some embodiments, the rubber stopper may be a chlorobutyl rubber stopper with a B2-40 coating. In some embodiments, the rubber stopper may include a plug portion. In some embodiments, the plug portion may include a Flurotec coating.

In some embodiments, the light-protective packaging may include solid black printing on its interior. In some embodiments, the kit may further including an IV bag. In some embodiments, the IV bag may be configured to block at least 85% of UV/visible light between 290 nm and 450 nm.

In some embodiments, the IV bag may include a light-protective sleeve. In some embodiments, the kit may further including IV tubing. In some embodiments, the IV tubing may be configured to block at least 85% of UV/visible light between 290 nm and 450 nm. In some embodiments, the IV tubing may include a light-protective sleeve.

Embodiments of the present disclosure may also include a method of administering plinabulin to a subject in need thereof, the method including providing plinabulin in a light-protected IV bag. Embodiments may also include administering plinabulin to a subject in need thereof through light-protected IV tubing. In some embodiments, the light-protected IV bag blocks at least 85% of UV/visible light between 290 nm and 450 nm. Any of the preceding embodiments directed to kits and formulations may be advantageously employed to perform the disclosed method.

In some embodiments, the light-protected IV tubing blocks at least 85% of UV/visible light between 290 nm and 450 nm. In some embodiments, the method of any one to 32. In some embodiments, providing the plinabulin may include injecting the plinabulin into the light-protected IV bag. In some embodiments, the plinabulin may be injected as a liquid formulation into D5W.

In some embodiments, the liquid formulation may include propylene glycol, and polyoxyl 15 hydroxystearates (PEG15-hydroxystearates). In some embodiments, the liquid formulation may include 60:40 (wt:wt) of propylene glycol and polyoxyl 15 hydroxystearates (PEG15-hydroxystearates). In some embodiments, the liquid formulation may include plinabulin, which may be at a concentration of about 40 mg/10 ml.

In some embodiments, providing the plinabulin may include injecting the plinabulin into a non-light-protected IV bag and then placing a light-protecting sleeve over the non-light-protected IV bag. In some embodiments, the method may include placing non-light-protected IV tubing within a light-protected sleeve to form a light-protected IV tubing.

### Light-Protective Vessel

In some embodiments, plinabulin is provided to care-givers as a liquid formulation. In some embodiments, the plinabulin liquid formulation comprises solvents polyoxyl 15 hydroxystearate and propylene glycol. In some embodiments, the solvent comprises 40% by weight polyoxyl 15 hydroxystearate and 60% by weight propylene glycol. In some embodiments, the concentration of plinabulin in liquid formulation is 40 mg/10 ml. It was discovered that such liquid formulations of plinabulin are extremely sensitive to light. Accordingly, in some embodiments, plinabulin liquid formulations are provided in a light-protective vessel.

In some embodiments, the light-protective vessel is configured to block, or reduce transmission of, UV/Visible light. In some embodiments, the light-protective vessel is configured to block, or reduce transmission of, UV/Visible light preferentially below a certain wavelength (e.g., below 520 nm). In some embodiments, the light-protective vessel is configured to block at least 99%, at least 98%, at least 97%, at least 96%, at least 95%, at least 94%, at least 93%, at least 92%, at least 91%, at least 90%, at least 85%, at least 80% UV/Visible light (in some embodiments, UV/Visible light below 520 nm), or ranges including and/or spanning the aforementioned values. In some embodiments, vessels used in packaging liquid preparations for parenteral and oral use should be made from material that is sufficiently transparent at some wavelengths to permit the visual inspection of the contents.

In some embodiments, the light-protective vessel is configured to block, or reduce transmission of, UV/Visible light below 580 nm. In some embodiments, the light-protective vessel is configured to block, or reduce transmission of, UV/Visible light below 520 nm. In some embodiments, the light-protective vessel is configured to block, or reduce transmission of, UV/Visible light below 450 nm. In some embodiments, the light-protective vessel is configured to block, or reduce transmission of, UV/Visible light below 400 nm. In some embodiments, the light-protective vessel is configured to block, or reduce transmission of, UV light below 315 nm. In some embodiments, the light-protective vessel is configured to block, or reduce transmission of, UV/light below 280 nm. In some embodiments, the light-protective vessel is configured to block, or reduce transmission of, light having a wavelength from about 100 nm to about 580 nm. In some embodiments, the light-protective vessel is configured to block, or reduce transmission of, light having a wavelength from about 100 nm to about 400 nm. In some embodiments, the light-protective vessel is configured to block, or reduce transmission of, light having a wavelength from about 290 nm to about 450 nm. In some embodiments, the light-protective vessel is configured to block, or reduce transmission of, light having a wavelength from about 100 nm to about 315 nm. In some embodiments, the light-protective vessel is configured to block, or reduce transmission of, light having a wavelength from about 100 nm to about 280 nm. In various embodiments, the foregoing reduction in light transmission at the specified wavelengths is reduced by at least 99%, 95%, 90%, 85%, 80%, 75%, 70%, 65%, or 60%. In some embodiments, the light-protective vessel is configured such that the maximum percentage of spectral transmission at any wavelength between 290 nm and 450 nm is less than 25%, 20%, 15%, 14%, 13%, 12%, 11%, 10%, 9%, 8%, 7%, 6%, or 5%.

In some embodiments, the light-protective vessel is an amber bottle. In some embodiments, the light-protective vessel is an amber polypropylene bottle. In some embodiments, the light-protective vessel is an amber glass vial. In some embodiments, the light-protective vessel is a borosilicate glass vial. In some embodiments, the light-protective vessel is a low actinic glass vial. In some embodiments, the light-protective vessel is a soda-lime glass vial. In some embodiments, the light-protective vessel is a single use vial.

In some embodiments, the light-protective vessel includes a coating. In some embodiments, the coating is an ethylene-acrylate coating. In some embodiments, the coating is a PTFE liner.

In some embodiments, the light-protective vessel includes a flip-off cap. In some embodiments, the light-protective vessel includes a polypropylene plug seal cap. In some embodiments, the light-protective vessel includes a rubber stopper. In some embodiments, the rubber stopper is a chlorobutyl rubber stopper with a B2-40 coating. In some embodiments, the rubber stopper includes a plug portion. In some embodiments, the plug portion includes a coating. In some embodiments, the coating is a Fluortec coating. In some embodiments, the light-protective vessel includes a rubber-lined cap. In some embodiments, the rubber-lined cap is a black phenolic rubber-lined cap.

### Light-Protective Packaging

In some embodiments, the light-protective vessel is additionally provided to care-givers in a light-protected packaging. In some embodiments, the light-protected packaging is a UV/Visible light blocking packaging. In some embodiments, the light-protected packaging is configured to block at least 99%, 98%, at least 97%, at least 96%, at least 95%, at least 94%, at least 93%, at least 92%, at least 91%, at least 90% UV/Visible light, at least 85% UV/Visible light, at least 80% UV/Visible light, or ranges including and/or spanning the aforementioned values.

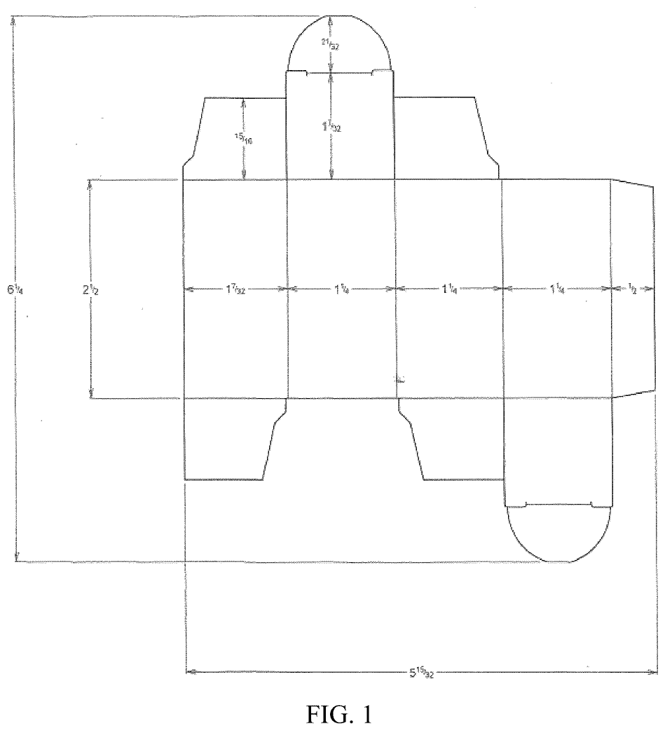

In some embodiments, the light-protective packaging includes a box (e.g., a cardboard box). In some embodiments, the box includes a coating that provides light protective properties. The coating may either be on the interior or exterior of the box. In some embodiments, the coating is printing on the cardboard. In some embodiments, the printing includes solid black printing on the interior of the box. FIG. 1 illustrates one embodiment of a box that can be used as described herein. FIG. 1 depicts a piece of cardboard cut in a shape that can be bent into a box shape. One side of the cardboard contains solid black printing. The cardboard can be folded into a box shape with the solid black printing being on the interior of the box. The light-protective vessel (e.g., amber vial) containing the plinabulin liquid formulation can then be placed inside the box. The light protection conferred by the box enhances that provided by the light-protective vessel.

In some embodiments, the light-protective vessel and packaging enables long-term storage of the plinabulin liquid formulation while the liquid formulation maintains less than 5.0%, less than 4.5%, less than 4.0%, less than 3.5%, less than 3.0%, less than 2.5% less than 2.0%, less than 1.5%, less than 1.0%, less than 0.5% w/w total impurities, or ranges including and/or spanning the aforementioned values. In some embodiments, the stored plinabulin liquid formulation includes less than 0.5% ether impurity. In some embodiments, the plinabulin liquid formulation includes less than 0.5% alcohol impurity. In some embodiments, the liquid formulation comprises less than 0.5% water.

### Light-Protective Administration Protocol

Due to the extreme sensitivity of plinabulin liquid formulations to light, it was also discovered that it is necessary to protect the liquid formulations throughout the administration protocol. Accordingly, in some embodiments, plinabulin is maintained in light-protected vessel throughout administration.

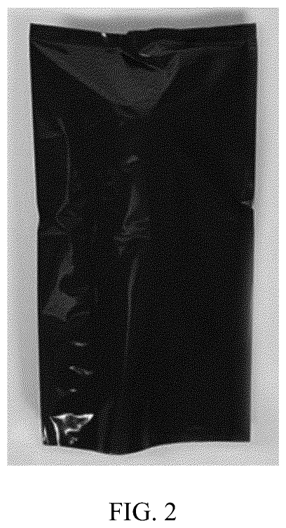

In some embodiments, the light-protective vessel is an IV bag configured to block UV/visible light as described above. In some embodiments, the IV bag is a translucent amber IV bag. In some embodiments, the IV bag is a dark amber bag. In some embodiments, the IV bag is a light amber bag. In some embodiments, the light-protective vessel is an IV bag covered by a light-protective sleeve. FIG. 2 illustrates an example of a light-protective sleeve for an IV bag.

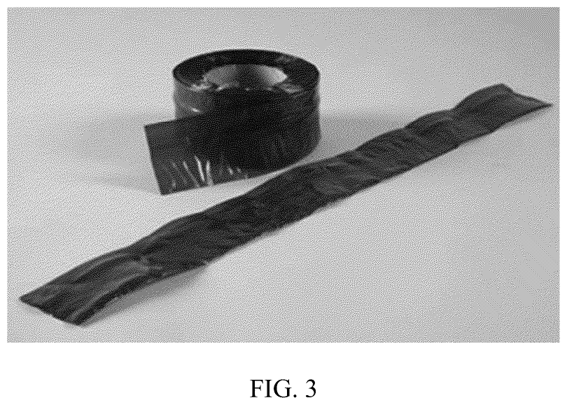

In some embodiments, the light-protective vessel is IV tubing configured to block UV/visible light as described above. In some embodiments, the IV tubing is a translucent amber IV tubing. In some embodiments, the IV tubing is a dark amber tubing. In some embodiments, the IV tubing is a light amber tubing. In some embodiments, the light-protective vessel is an IV tubing covered by a light-protective sleeve. FIG. 3 illustrates an example of a light-protective sleeve for IV tubing.

Some embodiments include adding the plinabulin liquid formulation contained in the light-protective vial described above to a light-protected IV bag. Some embodiments include administering plinabulin from the light-protected IV bag to a subject in need thereof by way of light-protected IV tubing. In some embodiments, the method includes gently mixing the light-protected IV bag several times to assure adequate mixing.

In some embodiments, the light-protected IV bag includes a D5W (dextrose 5% in water) liquid formulation prior to adding the plinabulin. In various embodiments, the IV bag includes 150 ml, 200 ml, 250 ml, 300 ml, 350 ml, 400 ml, 450 ml, 500 ml of D5W, or ranges including and/or spanning the aforementioned value. In some embodiments after adding the plinabulin liquid formulation from the light-protected vial, the light-protected IV bag includes a plinabulin liquid formulation at a concentration from about 0.08 mg/ml to about 0.2 mg/ml.

In some embodiments, the method of administering plinabulin to a subject in need thereof includes providing plinabulin in a light-protected IV bag and administering plinabulin to a subject in need thereof through light-protected IV tubing. In some embodiments, the light-protected IV bag blocks at least 85% UV/visible light between 290 nm and 450 nm. In some embodiments, the light-protected IV tubing blocks at least 85% UV/visible light between 290 nm and 450 nm. In some embodiments, providing the plinabulin comprises injecting the plinabulin into the light-protected IV bag. In some embodiments, providing the plinabulin comprises injecting the plinabulin into a non-light-protected IV bag and then placing a light-protecting sleeve over the non-light-protected IV bag. In some embodiments, the plinabulin is injected as a liquid formulation into D5W in the light protected IV bag. In some embodiments, the liquid formulation comprises propylene glycol, and polyoxyl 15 hydroxystearates (PEG15-hydroxystearates). In some embodiments, the liquid formulation comprises 60:40 (wt:wt) of propylene glycol and polyoxyl 15 hydroxystearates (PEG15-hydroxystearates). In some embodiments, the liquid formulation comprises plinabulin is at a concentration of about 40 mg/10 ml. In some embodiments, the method may further include placing non-light-protected IV tubing within a light-protected sleeve to form the light-protected IV tubing.

In some embodiments, the plinabulin liquid formulation in the IV bag may be administered within 6 hours of preparation. In some embodiments, the plinabulin is kept at room temperature during administration to a subject in need thereof.

### Stable Plinabulin Composition

In some embodiments, the plinabulin liquid formulation in a light-protected vessel is a stable formulation of plinabulin. In some embodiments, the stable formulation is stable for a period of storage of at least about 1 week, at least about 2 weeks, at least about 3 weeks, at least about 1 month, at least about 2 months, at least about 3 months, at least about 4 months, at least about 5 months, at least about 6 months, at least about 7 months, at least about 8 months, at least about 9 months, at least about 12 months, at least about 18 months, at least about 24 months, at least about 36 months, or at least about 48 months. In some embodiments, the stable formulation retains at least about 99%, 95%, 90%, 80%, 70%, or 60% plinabulin strength after the period of storage.

In some embodiments, the stable formulations and stable pharmaceutical formulations described herein are stable when stored for a period of time at any one or more of −20° C., −10° C., 0° C., 5° C., 10° C., 25° C., 20° C., 30° C., 40° C., or ranges including and/or spanning the aforementioned values.

### Use and Methods

Some embodiments related to a method of halting or reversing a progressive cancer in a subject. In some embodiments, the method comprises administering Plinabulin to the subject. Some embodiments relate to a method of inhibiting progression of a cancer. Further uses of plinabulin include those described in U.S. Pat. Nos. 7,919,497; 10,238,650; 10,155,748; 10,076,518; and 10,596,169; and PCT Publication Nos. WO 2016/130839; WO 2017/214052; WO 2018/144764; WO 2018/169887; WO 2019/147615; WO 2019/147615; WO 2019/152530; WO 2020/037285; WO 2021/076485; and WO 2021/225908; all of which are incorporated herein by reference in their entirety.

In some embodiments, the treatment schedule includes administration of Plinabulin once every 1 week, 2 weeks, 3 weeks, 4 weeks, 5 weeks, 6 weeks, 7 weeks, or 8 weeks. In some embodiments, the treatment schedule includes administration of Plinabulin two times every 1 week, 2 weeks, 3 weeks, 4 weeks, 5 weeks, 6 weeks, 7 weeks, or 8 weeks. In some embodiments, the treatment schedule includes administration of Plinabulin once every 1 week in a treatment cycle of 1 week, 2 weeks, 3 weeks, 4 weeks, 5 weeks, 6 weeks, 7 weeks, or 8 weeks. In some embodiments, the treatment schedule includes administration of Plinabulin twice every 1 week in a treatment cycle of 1 week, 2 weeks, 3 weeks, 4 weeks, 5 weeks, 6 weeks, 7 weeks, or 8 weeks. In some embodiments, the treatment schedule includes administration of Plinabulin on day 1, day 8, and day 15 of a 21-day treatment cycle.

The treatment cycle can be repeated as long as the regimen is clinically tolerated. In some embodiments, the treatment cycle for Plinabulin is repeated for n times, wherein n is an integer in the range of 2 to 30. In some embodiments, n is 2, 3, 4, 5, 6, 7, 8, 9, or 10. In some embodiments, a new treatment cycle can occur immediately after the completion of the previous treatment cycle. In some embodiments, a new treatment cycle can occur a period of time after the completion of the previous treatment cycle. In some embodiments, a new treatment cycle can occur after 1 week, 2 weeks, 3 weeks, 4 weeks, 5 weeks, 6 weeks, or 7 weeks after the completion of the previous treatment cycle.

In some embodiments, the method comprises administering plinabulin at a dose from about 5 mg/m² to 150 mg/m². In some embodiments, plinabulin is administered at a dose that is greater than 30 mg/m². In some embodiments, plinabulin is administered at a dose of about 40 mg/m².

In some embodiments, the plinabulin is administered on day 1 of a 14 day dosing cycle. In some embodiments, plinabulin is administered on day 1 of a 21 day dosing cycle.

In some embodiments, the plinabulin liquid formulation is co-administered with one or more G-CSF drug. In some embodiments, the one or more G-CSF drug is paclitaxel.

### Kits

Some embodiments include kits comprising one or more containers.

In some embodiments, the container comprises a plastic or glass, or combination thereof, including but not limited to any one or more of a plastic or glass used by those of skill in the art given the teachings herein. In some embodiments, the portion of the container to which a liquid formulation is exposed is made of an amber glass. In some embodiments, the container is tinted amber. In some embodiments, the container is opaque or translucent to protect the formulation from light or other forms of radiation, with portions that are transparent or translucent. In some embodiments, the dosage kit and packaging are selected from the group consisting of amber vials to protect from light and stoppers with specialized coatings to improve shelf-life stability.

In some embodiments, the container is a vial. In some embodiments, the vial comprises plinabulin in the amount of about 1 mg, 2 mg, 5 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg 190 mg, 200 mg or ranges including and/or spanning the aforementioned values.

In some embodiments, the container is an IV bag. In some embodiments, the IV bag includes a dark amber IV cover. In some embodiments, the IV bag is UV/visible light blocking. In some embodiments, the IV bag blocks 95% of UV/visible light. In some embodiments, IV bag is opaque. In some embodiments, the IV bag is transparent but masks differences in IV fluid color.

In some embodiment, the kit further comprises an IV tubing cover. In some embodiments, the IV tubing cover is an amber IV tubing cover. In some embodiments, amber IV tubing cover provides additional protection for the plinabulin composition or pharmaceutical composition.

In some embodiments, the containers described herein are in a secondary packaging. In some embodiments, the secondary packaging that is configured to preserve the stability of the therapeutic agent relative to the stability of the therapeutic agent prior to packaging. In some variations, the secondary packaging is designed to exclude or reduce exposure of the formulation to light. In some embodiments, the secondary packaging is designed to exclude or reduce exposure of the formulation to one or more components of the air, such as oxygen. In some variations, the container is an ampule, vial, or prefilled syringe.

In some embodiments, the kit may include plinabulin for injection in a single use amber glass vial with a chlorobutyl rubber stopper and Flurotec coating on the plug portion of the stopper and flip-off cap.

In some embodiments, the formulations described herein are provided in one or more unit dose forms, wherein the unit dose form contains an amount of a liquid plinabulin formulations described herein that is effective to treat or prevent the disease or condition for which it is being administered.

In some embodiments, the unit dose form is prepared in the concentration at which it will be administered. In some variations, the unit dose form is diluted prior to administration to a subject.

In a further aspect, provided herein are kits comprising one or more unit dose forms as described herein. In some embodiments, the kit comprises one or more of packaging and instructions for use to treat one or more diseases or conditions. In some embodiments, the kit comprises a diluent which is not in physical contact with the formulation or pharmaceutical formulation. In some embodiments, the kit comprises any of one or more unit dose forms described herein in one or more sealed vessels. In some embodiments, the kit comprises any of one or more sterile unit dose forms.

In some embodiments, the kits may contain a G-CSF drug. Granulocyte-colony stimulating factor (G-CSF) refers to compounds or factors that stimulate proliferation, differentiation, commitment and end cell functional activation of granulocytes in an animal, including a human subject. G-CSF compounds include but are not limited to filgrastim and pegfilgrastim. Examples of G-CSF that are commercially available include but are not limited to Neupogen® (Amgen), Tevagrastim® (Teva), Biograstim® (CT Arzneimittel), Ratiograstim® (Ratiopharm GmbH)), Zarxio® (Sandoz GmbH), Filgrastim Hexal® (Hexal AG), Neulasta® (Amgen), Granocyte® and Neutrogin® (Chugai), and Neu-up® (Kyowa Hakko). G-CSF is often given to manage chemotherapy-induced severe neutropenia. G-CSF such as pegfilgrastim is a colony-stimulating factor that acts on hematopoietic cells by binding to specific cell surface receptors, thereby stimulating proliferation, differentiation, commitment, and end cell functional activation.

### Examples

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### Example 1

This comparative example reports a plinabulin stress test of solid plinabulin showing relative stability to light storage for 30 days. Table 1 reports the results of impurity analysis upon 30 days of exposure of plinabulin to light. Table 2 reports the results impurity analysis of a control sample stored under dark conditions. The results indicate that total impurities only increased from 0.3% to 0.6% after 30-days exposure to light.

TABLE 1

| Storage Conditions | | | Photo Sample | | |
|---|---|---|---|---|---|
| Test Items | | 0 days | 5 days | 10 days | 30 days |
| Assay by HPLC | | 98.9% w/w | 99.37% w/w | 99.36% w/w | 99.17% w/w |
| Impurity | Amide Impurity | 0.02% | 0.02% | 0.02% | 0.02% |
| by | De-Carboxylate | 0.1% | 0.09% | 0.08% | 0.09% |
| HPLC | Impurity | | | | |
| | Ether Impurity | 0.02% | 0.02% | 0.02% | 0.02% |
| | Alcohol Impurity | 0.02% | 0.02% | 0.02% | 0.02% |
| | Cyano Impurity | 0.1% | 0.11% | 0.12% | 0.11% |
| | Impurity-489-1 | 0.1% | 0.10% | 0.07% | 0.06% |
| | Impurity-489-2 | 0.1% | 0.05% | 0.05% | 0.05% |
| | Other Unspecified | 0.02% | 0.02% | 0.02% | 0.1% |
| | Impurity | | | | |
| | Total Impurities | 0.3% | 0.3% | 0.27% | 0.60% |

| | | | | |
|---|---|---|---|---|
| Water | 0.1% | 0.05% | 0.05% | 0.05% |

TABLE 2

| Storage Conditions | | | Control Sample | | |
|---|---|---|---|---|---|
| Test Items | | 0 days | 5 days | 10 days | 30 days |
| Appearance | | Yellow | Yellow | Yellow | Yellow |
| | | Solid | Solid | Solid | Solid |
| Assay by HPLC | | 98.9% w/w | 98.42% w/w | 99.33% w/w | 99.29% w/w |
| Impurity | Amide Impurity | 0.02% | 0.02% | 0.02% | 0.02% |
| by | De-Carboxylate | 0.1% | 0.09% | 0.09% | 0.09% |
| HPLC | Impurity | | | | |
| | Ether Impurity | 0.02% | 0.02% | 0.02% | 0.02% |
| | Alcohol Impurity | 0.02% | 0.02% | 0.02% | 0.02% |
| | Cyano Impurity | 0.1% | 0.11% | 0.12% | 0.11% |
| | Impurity-489-1 | 0.1% | 0.16% | 0.12% | 0.15% |
| | Impurity-489-2 | 0.1% | 0.02% | 0.02% | 0.02% |
| | Other Unspecified | 0.02% | 0.02% | 0.02% | 0.05% |
| | Impurity | | | | |
| | Total Impurities | 0.3% | 0.36% | 0.32% | 0.36% |

| | | | | |
|---|---|---|---|---|
| Water | 0.1% | 0.05% | 0.05% | 0.05% |

### Example 2

This example presents results from a study performed to evaluate the photostability of plinabulin liquid formulation, 4 mg/mL (40 mg/10 mL), in 40% by weight polyoxyl 15 hydroxystearate and 60% by weight propylene glycol with different packaging configurations. Table 3 describes the packaging used and the results of the study are presented in Table 4.

TABLE 3

| | |
|---|---|
| Product | Plinabulin liquid formulation, |
| Name/Strength | 4 mg/ml (40 mg/10 ml) |
| Drug Product Vial | Depoyrogenerated Vials: Vial, 10 ml 20 mm |
| Description | Finished Nipro Type I, Amber |
| (Primary | Pii Material Code 9807-N33, Pii Lot# 18-0240 |
| Packaging) | Sterilized Stoppers: Stoppers, 20 mm |
| | (S10-F451) Gray B2-40 Coating |
| | Pii Material Code 9527-W20, Pii Lot# 19-0672 |
| | Seals: West 20 mm Flip-Off Truedge |
| | Lacquered Clear 4020 Dark Blue Matte Top Button |
| | Pii Material Code 9170-W20, Pii Lot# 19-0900 |
| IFC Carton | Inner Folding Carton: 1-¼ × ¼ × ⅖ |
| Description | .015SBS/C/1/S Printed Black on Inside |
| (Secondary | Material Code: 9963-C49 |
| Packaging) | |

The following configurations were tested:
1) Unexposed finished product in primary packaging (Control).
2) Finished product in primary packaging wrapped in aluminum foil and exposed to UV/Vis light (Dark Control).
3) Finished product in primary packaging (amber vial) exposed to UV/Vis light.
4) Finished product vial in secondary packing (IFC carton) exposed to UV/Vis light.

Light intensity exposure was determined on the day of testing and the actual hours tested were noted. The photostability study was performed in accordance with VP 5740 and ICH Q1B: Photostability Testing of New Drug Substances and Products (November 1996).

Samples of Plinabulin liquid formulation in primary packaging (amber vial) and secondary packaging (IFC carton) were exposed to UV/Vis light providing an overall illumination of 1.21 million luxhours and an integrated near UV energy of 326.6 watt-hours/m², meeting the criteria of an overall illumination of not less than 1.2 million lux-hours and an integrated near UV energy of not less than 200 watt-hours/m², per ICH Q1B Option 1. An unexposed control sample was evaluated for information only. Finished product samples wrapped in aluminum foil (dark controls) were exposed to light to evaluate the contribution of thermally induced change to the total observed change. Photostability was determined on the basis of appearance, identification by retention time, identification by UV spectrum match/PDA, moisture content, assay, impurities, container content and particulate matter.

At the conclusion of the photostability study, the samples were evaluated for any changes in the physical properties of the sample liquid formulation (i.e., appearance) and were also tested for identification by retention time, identification by UV spectrum match/PDA, moisture content, assay, impurities, container content and particulate matter.

TABLE 4

| Test and Test | | | |
|---|---|---|---|
| Method | Acceptance Criteria | Results | |
| Appearance | Clear yellow liquid formulation | Control | |
| | in a 10 ml amber glass vial with | Clear Yellow liquid formulation, | |
| | grey stopper and blue flip-off | no particles were observed | |
| | cap | Dark Control | |
| | | Clear yellow liquid formulation, | |
| | | no particles were observed | |
| | | Primary Packaging | |
| | | Clear yellow liquid formulation, | |
| | | no particles were observed | |
| | | Secondary Packaging | |
| | | Clear yellow liquid formulation, | |
| | | no particles were observed | |
| Identification by | The Retention Time of the | Sample | RT |
| Retention Time (RT)/ | major peak in the sample | Control | 11.4 |
| HPLC | chromatogram corresponds to | Dark Control | 11.4 |
| | that in the standard | Primary Packaging | 11.4 |
| | chromatogram | Secondary Packaging | 11.4 |
| Identification by UV | The UV spectrum of the major | Sample | Match Angle |
| Spectrum Match/ | peak in the sample | Control | 0.008 |
| PDA | chromatogram corresponds to | Dark Control | 0.010 |
| TM000280 | that in the standard | Primary Packaging | 0.019 |
| (TM 14-0126) | chromatogram | Secondary Packaging | 0.012 |
| | | Sample | Match Threshold |
| | | Control | 1.002 |
| | | Dark Control | 1.002 |
| | | Primary Packaging | 1.003 |
| | | Secondary Packaging | 1.002 |
| Moisture Content | % w/w: Report | Sample | % w/w |
| Current USP, 921., | | Control | 0.17 |
| Method Ia | | Dark Control | 0.18 |
| | | Primary Packaging | 0.17 |
| | | Secondary Packaging | 0.18 |
| HPLC Assay | Label Claim: | Sample | % LC |
| TM000280 | 90.0%-110.0% | Control | 98.4 |
| (TM 14-0126) | | Dark Control | 97.8 |
| | | Primary Packaging | 88.9 |
| | | Secondary Packaging | 97.6 |
| | mg/ml: 3.6 mg/ml-4.4 mg/ml | Sample | mg/ml |
| | | Control | 3.9 |
| | | Dark Control | 3.9 |
| | | Primary Packaging | 3.9 |
| | | Secondary Packaging | 3.9 |
| Impurities (HPLC) | NPI-2489 | Sample | % w/w |
| TM000280 | NMT 0.5% w/w | Control | <0.10 |
| (TM 14-0126) | | Dark Control | <0.10 |
| | | Primary Packaging | 12.8 |
| | | Secondary Packaging | <0.10 |
| | NPI-2601 | Sample | % w/w |
| | NMT 0.5% w/w | Control | <0.10 |
| | | Dark Control | <0.10 |
| | | Primary Packaging | <0.10 |
| | | Secondary Packaging | <0.10 |
| | Any Individual Unknown | Sample | % w/w |
| | Impurity ≥0.1% | Control | <0.10 |
| | NMT 0.2% w/w | Dark Control | <0.10 |
| | | Primary Packaging | 0.10 |
| | | Secondary Packaging | <0.10 |
| | Total Impurities: | Sample | % w/w |
| | NMT 1.0% w/w | Control | 0.0 |
| | | Dark Control | 0.0 |
| | | Primary Packaging | 12.9 |
| | | Secondary Packaging | 0.0 |

Results for assay and individual and total impurities did not meet the acceptance criteria for primary packaging (amber vial), suggesting that primary packaging does not provide adequate protection from light. All other results for control, dark control and secondary packaging (IFC carton) met the acceptance criteria as defined.

Based on the results generated it can be concluded that placing an amber vial in IFC carton provides sufficient protection from light for Plinabulin liquid formulation 4 mg/mL (40 mg/10 mL).

### Example 3

This example reports results from a study performed to determine the effect of light exposure on appearance, ID, assay and impurities of Plinabulin liquid formulation in infusion lines after 1:20 and 1:50 dilution of a 4 mg/ml liquid formulation (40% by weight polyoxyl 15 hydroxystearate and 60% by weight propylene glycol) in a 5% Dextrose in Water (D5W) infusion bag. The target illuminance was 1000 lux on the infusion line as per ANSI/IES RP-29-20 (Recommended Practice: Lightning Hospital and Healthcare Facilities, an American National Standard).

The study utilized 200-500 mL non-PVC bags at dilution ratios of 1:20 and 1:50 at various time intervals. Plinabulin liquid formulation, 4 mg/mL, was separately diluted in D5W at the 1:20 and 1:50 dilution levels. The dilution was performed using a clear vial of Plinabulin liquid formulation, 4 mg/mL. Samples were pulled from the infusion line after light exposure at the following time intervals: 3 min, 5 min, 7 min, 11 min, 14 min, and 22 min. A control sample (T=0) was pulled from the exit port of the D5W bag containing the diluted Plinabulin drug product at the 0 minute time point.

### Materials and Methods

Plinabulin liquid formulation 4 mg/mL: 10 mL amber vials. D5W liquid formulation: 200-500 mL bags of sterile D5W in non-PVC bags, B. Braun, Lot #J9N016. Dark Amber D5W bag covers: Health Care Logistics Item #7589D. Amber IV tubing sleeve: Amber Tubing Cover Roll cut to 72 and 103 inches, Health. Care Logistics Item #19645. Liquid formulation Set with DUO-VENT spike, polyethylene lined tubing 103 inches, Baxter Item #2C8875. Light Intensity Meter ID #2359506.

### Sample Procedure

All operations were carried out in the dark room; the drug liquid formulation was not heated or refrigerated once it was diluted. All D5W non-PVC IV bags used in the study were from the same manufacturer/supplier and from the same manufacturer's lot. Infusion bags were prepared for the study. Plinabulin liquid formulation, 4 mg/mL was diluted 1:20 or 1:50 with D5W in a non-PVC IV bag to produce diluted IV infusion liquid formulations (infusion bag).

### Preparation of Infusion Liquid Formulation

The IV line volumes were measured and recorded:7 mL for 72 inch line; and 10 mL for 103 inch line. One D5W IV bag was taken and a light protective sleeve was placed over D5W IV bag and tubing set. The required volume was withdrawn from a vial using a Luer Lock syringe. The required volume of drug product was immediately diluted into the light protected D5W bag that contains 200-500 mL per label claim of D5W to the required dilution of 1:20 or 1:50. The IV bag was gently inverted several times to assure adequate mixing. The resultant dilution was visually confirmed to be a clear liquid formulation. TA Time 0 sample was pulled from each prepared bag and transferred to a labeled sample container and protected from light. Individual infusion lines were filled for each time point from the prepared infusion bags and these samples were used for testing the effects of light exposure on the naked IV infusion line and on the IV infusion line with amber sleeve protection. The filled infusion lines were protected from light prior to exposure to white light and labeled properly.

### Exposure Procedure

All operations were carried out in the dark room. A white light was installed in the dark room. The light intensities were measured and recorded at the beginning of the sample exposure. The target illuminance was 1000 lux on the exposure site (infusion line) as per ANSI/IES RP-29-20. Table 5 describes light intensity measurements.

TABLE 5
Illuminance (lux)

| 72 Inch IV Line | | | | 103 Inch IV Line | | | |
|---|---|---|---|---|---|---|---|
| 1:20 | | 1:50 | | 1:20 | | 1:50 | |
| Naked | Amber | Naked | Amber | Naked | Amber | Naked | Amber |
| 1035 | 1035 | 1020 | 1030 | 1025 | 1035 | 1030 | 1015 |

The samples of Plinabulin Injection after dilution in D5W in a non-PVC bag at dilution ratios of 1:20 and 1:50, after filling into a naked infusion line and an infusion line wrapped by amber sleeve at ambient conditions, were exposed to light for the time intervals by turning on the white light for the predetermined time.

### Testing Plan

Other than the T=0 samples, the samples were exposed in the light under the conditions described above. Light protection conditions on the filled infusion lines: Naked line (no protection): 72 inches; Naked line (no protection): 103 inches; Infusion line with Amber sleeve: 72 inches; and Infusion line with Amber sleeve: 103 inches. The entire contents of each IV line was transferred to a pre-labelled amber sample container and stored at room temperature, protected from light, until analytical testing. Samples were tested within 6 hours of product dilution. The samples were tested as described herein.

### Test Methods and Acceptance Criteria

The impurity/degradants were assayed by HPLC. During the infusion line light exposure study, samples were withdrawn from the individual infusion line or from the exit port, as applicable, and directly tested without further dilution and/or filtration. The acceptance criteria and results of the infusion line light exposure study are presented in Table 6 (1:20 Dilution) and Table 7 (1:50 Dilution).

TABLE 6

| | Sample | Acceptance | | |
|---|---|---|---|---|
| Test | Volume | Criteria | Results | |
| Initial Vial | Vial | Clear yellow | Clear yellow liquid formulation in a 10 ml | |
| Appearance | | liquid | amber glass vial with grey stopper and | |
| | | formulation in a | blue flip-off cap | |
| | | 10 ml amber | | |
| | | glass vial with | | |
| | | grey stopper | | |
| | | and blue flip- | | |
| | | off cap | | |
| Appearance of | Entire | Report | Naked Line 72″ | |
| Sample | Sample | | Clear light yellow liquid formulation for | |
| | | | all time points: 0 min, 1 min, 2 min, 3 min, | |
| | | | 5 min, 7 min, 11 min, 14 min, and 22 min. | |
| | | | Naked Line 103″ | |
| | | | Clear light yellow liquid formulation for | |
| | | | all time points: 0 min, 1 min, 2 min, 3 min, | |
| | | | 5 min, 7 min, 11 min, 14 min, and 22 min. | |
| | | | Infusion Line with Amber Sleeve 72″ | |
| | | | Clear light yellow liquid formulation for | |
| | | | all time points: 0 min, 1 min, 2 min, 3 min, | |
| | | | 5 min, 7 min, 11 min, 14 min, and 22 min. | |
| | | | Infusion Line with Amber Sleeve 72″ | |
| | | | Clear light yellow liquid formulation for | |
| | | | all time points: 0 min, 1 min, 2 min, 3 min, | |
| | | | 5 min, 7 min, 11 min, 14 min, and 22 min. | |
| Assay | All of the | | Naked Line 72″ | |
| | liquid in | | Time 0 | 97.2 |
| | the IV line | | 1 min | 96.8 |
| | | | 2 min | 96.6 |
| | | | 3 min | 96.3 |
| | | | 5 min | 96.0 |
| | | | 7 min | 95.5 |
| | | | 11 min | 94.6 |
| | | | 14 min | 94.0 |
| | | | 22 min | 91.6 |
| | | | Naked Line 103″ | |
| | | | Time 0 | 98.1 |
| | | | 3 min | 97.1 |
| | | | 5 min | 96.1 |
| | | | 7 min | 95.6 |
| | | | 11 min | 94.9 |
| | | | 14 min | 94.3 |
| | | | 22 min | 92.8 |
| | | | Infusion Line with Amber Sleeve 72″ | |
| | | | Time 0 | 97.0 |
| | | | 3 min | 96.8 |
| | | | 5 min | 96.7 |
| | | | 7 min | 96.6 |
| | | | 11 min | 96.5 |
| | | | 14 min | 96.6 |
| | | | 22 min | 96.4 |
| | | | Infusion Line with Amber Sleeve 103″ | |
| | | | Time 0 | 97.4 |
| | | | 3 min | 97.3 |
| | | | 5 min | 97.2 |
| | | | 7 min | 97.3 |
| | | | 11 min | 97.1 |
| | | | 14 min | 97.1 |
| | | | 22 min | 96.9 |
| Impurities | Use Assay | Impurity | Total Impurities Naked Line 72″ | |
| | Sample | Limit | Time 0 | 0.0 |
| | | NPI-2489 | 1 min | 0.3 |
| | | 0.5% | 2 min | 0.7 |
| | | NPI-2601 | 3 min | 1.0 |
| | | 0.5% | 5 min | 1.5 |
| | | Unknown | 7 min | 2.1 |
| | | 0.2% | 11 min | 3.0 |
| | | Total Imp. | 14 min | 3.7 |
| | | 1.0% | 22 min | 6.3 |

| Total Impurities Naked Line 103″ | |
|---|---|
| Time 0 | 0.0 |
| 3 min | 1.0 |
| 5 min | 1.9 |
| 7 min | 2.7 |
| 11 min | 3.5 |
| 14 min | 4.4 |
| 22 min | 5.9 |
| Total Impurities Infusion Line with | |
| Amber Sleeve 72″ | |
| Time 0 | 0.0 |
| 3 min | 0.0 |
| 5 min | 0.0 |
| 7 min | 0.1 |
| 11 min | 0.1 |
| 14 min | 0.2 |
| 22 min | 0.2 |
| Total Impurities Infusion Line with | |
| Amber Sleeve 103″ | |
| Time 0 | 0.0 |
| 3 min | 0.0 |
| 5 min | 0.0 |
| 7 min | 0.1 |
| 11 min | 0.1 |
| 14 min | 0.2 |
| 22 min | 0.3 |

TABLE 7

| | Sample | Acceptance | | |
|---|---|---|---|---|
| Test | Volume | Criteria | Results | |
| Initial Vial | Vial | Clear yellow liquid | Clear yellow liquid formulation | |
| Appearance | | formulation in a | in a 10 ml amber glass vial with | |
| | | 10 ml amber glass vial | grey stopper and blue flip-off | |
| | | with grey stopper | cap | |
| | | and blue flip-off | | |
| | | cap | | |
| Heat-Treated Vial | Vial | Report, if | N/A | |
| Appearance (if | | applicable. | | |
| applicable Visual) | | | | |
| Appearance of | Entire | Report | Naked Line 72″ | |
| Sample | Sample | | Clear light yellow liquid | |
| | | | formulation for all time points: | |
| | | | 0 min, 1 min, 2 min, 3 min, | |
| | | | 5 min, 7 min, 11 min, 14 min, | |
| | | | and 22 min. | |
| | | | Naked Line 103″ | |
| | | | Clear light yellow liquid | |
| | | | formulation for all time points: | |
| | | | 0 min, 1 min, 2 min, 3 min, | |
| | | | 5 min, 7 min, 11 min, 14 min, | |
| | | | and 22 min. | |
| | | | Infusion Line with Amber | |
| | | | Sleeve 72″ | |
| | | | Clear light yellow liquid | |
| | | | formulation for all time points: | |
| | | | 0 min, 1 min, 2 min, 3 min, | |
| | | | 5 min, 7 min, 11 min, 14 min, | |
| | | | and 22 min. | |
| | | | Infusion Line with Amber | |
| | | | Sleeve 72″ | |
| | | | Clear light yellow liquid | |
| | | | formulation for all time points: | |
| | | | 0 min, 1 min, 2 min, 3 min, | |
| | | | 5 min, 7 min, 11 min, 14 min, | |
| | | | and 22 min. | |
| Assay | All of the | | Naked Line 72″ | |
| | liquid in the | | Time 0 | 100.3 |
| | IV line | | 1 min | 98.8 |
| | | | 2 min | 99.2 |
| | | | 3 min | 98.4 |
| | | | 5 min | 97.2 |
| | | | 7 min | 95.7 |
| | | | 11 min | 94.5 |
| | | | 14 min | 94.0 |
| | | | 22 min | 91.5 |

| Naked Line 103″ | |
|---|---|
| Time 0 | 97.3 |
| 3 min | 96.7 |
| 5 min | 96.6 |
| 7 min | 95.6 |
| 11 min | 95.2 |
| 14 min | 94.9 |
| 22 min | 93.5 |
| Infusion Line with Amber | |
| Sleeve 72″ | |
| Time 0 | 98.8 |
| 3 min | 98.4 |
| 5 min | 98.7 |
| 7 min | 98.3 |
| 11 min | 99.0 |
| 14 min | 98.5 |
| 22 min | 97.6 |
| Infusion Line with Amber | |
| Sleeve 103″ | |
| Time 0 | 97.1 |
| 3 min | 97.2 |
| 5 min | 96.9 |
| 7 min | 97.0 |
| 11 min | 96.8 |
| 14 min | 96.8 |
| 22 min | 96.7 |

| | | | | |
|---|---|---|---|---|
| Impurities | Use Assay | Impurity | Limit | Total Impurities Naked Line |
| | Sample | NPI-2489 | 0.5% | 72″ |

| | | | |
|---|---|---|---|
| NPI-2601 | 0.5% | Time 0 | 0.0 |
| Unknown | 0.2% | 1 min | 0.4 |
| Total Imp. | 1.0% | 3 min | 1.3 |
| | | 5 min | 2.4 |
| | | 7 min | 3.6 |
| | | 11 min | 5.0 |
| | | 14 min | 6.0 |
| | | 22 min | 8.7 |

| Total Impurities Naked Line | |
|---|---|
| 103″ | |
| Time 0 | 0.0 |
| 3 min | 0.8 |
| 5 min | 1.0 |
| 7 min | 1.7 |
| 11 min | 2.5 |
| 14 min | 2.9 |
| 22 min | 4.6 |
| Total Impurities Infusion | |
| Line with Amber Sleeve 72″ | |
| Time 0 | 0.0 |
| 3 min | 0.0 |
| 5 min | 0.0 |
| 7 min | 0.1 |
| 11 min | 0.2 |
| 14 min | 0.2 |
| 22 min | 0.3 |
| Total Impurities Infusion | |
| Line with Amber Sleeve 103″ | |
| 3 min | 0.0 |
| 5 min | 0.0 |
| 7 min | 0.1 |
| 11 min | 0.2 |
| 14 min | 0.3 |
| 22 min | 0.4 |

Results for the NPI-2489 impurity did not meet the acceptance criteria of NMT 0.5% for the naked IV infusion line at 72 and 103 inches, with the exception of the 1 minute time point at 72 inches. All other results met the defined acceptance criteria. Based on the data from this study it can be concluded that results generated using the naked IV infusion line do not meet the specification acceptance criteria for the NPI-2489 impurity and that placing an amber IV tubing sleeve on the infusion line provides sufficient protection from light for up to 22 minutes.

### Example 4

In this comparative example, it was seen that solid plinabulin is not light sensitive. Table 8 illustrates the stability of solid plinabulin in a light sensitivity study.

TABLE 8

| | | | | Minimum of 1.2 million |
|---|---|---|---|---|
| | | | | LUX hours and 200 watts/ |
| Attributes | Specifications | Time 0 | Dark Control | square meter |
| Appearance | Yellow Solid | Yellow Solid. | Yellow Solid. | Yellow Solid. |
| | No visible | No visible | No visible | No visible |
| | contaminants | contaminants | contaminants | contaminants |
| KF | Report | 5.1% | 5.1% | 5.0% |
| FTIR | Must conform to | Conforms to | Conforms to | Conforms to |
| | FT2-0926 | FT2-0926 | FT2-0926 | FT2-0926 |
| HPLC | ≥95% AUC | 99.2% AUC | 99.3% AUC | 99.4% AUC |
| (% AUC) | at 214 nm | at 214 nm | at 214 nm | at 214 nm |
| | No single impurity | No single impurity | No single impurity | No single impurity |
| | >1% AUC at 214 nm | >1% AUC at 214 nm | >1% AUC at 214 nm | >1% AUC at 214 nm |
| | Report any impurity > | 0.21% @rrt 0.86 | 0.21% @rrt 0.86 | 0.02% @rrt 0.40 |
| | 0.02% at 214 nm by rrt | 0.36% @rrt 0.96 | 0.33% @rrt 0.97 | 0.05% @rrt 0.49 |
| | Report % AUC | 0.06% @rrt 1.14 | 0.06% @rrt 1.17 | 0.02% @rrt 0.68 |
| | at 250 nm | 0.05% @rrt 1.18 | 0.04% @rrt 1.27 | 0.08% @rrt 0.86 |
| | Report % AUC | 0.02% @rrt 1.19 | 99.5% AUC | 0.29% @rrt 0.97 |
| | at 370 nm | 0.02% @rrt 1.28 | at 250 nm | 0.05% @rrt 1.17 |
| | | 99.4% AUC | 99.4% AUC | 0.02% @rrt 1.27 |
| | | at 250 nm | at 370 nm | 99.4% AUC |
| | | 99.4% AUC | | at 250 nm |
| | | at 370 nm | | 99.5% AUC |
| | | | | at 370 nm |

What is claimed is:

1. A vessel comprising:
a liquid formulation comprising plinabulin, propylene glycol, and polyoxyl 15 hydroxystearates (PEG15-hydroxystearates),
wherein the vessel is configured to block at least 85% of UV/visible light between 290 nm and 450 nm.

2. The vessel of claim 1, wherein the vessel is a single use amber glass vial.

3. The vessel of claim 2, wherein the amber glass vial further comprises a rubber stopper and a flip-off cap.

4. The vessel of claim 3, wherein the rubber stopper is a chlorobutyl rubber stopper with a B2-40 coating.

5. The vessel of claim 3 or 4, wherein the rubber stopper further comprises a plug portion, wherein the plug portion comprises a Flurotec coating.

6. The vessel of claim 1, wherein the vessel is an IV bag.

7. The vessel of claim 3, wherein the vessel is an IV bag covered by a light-protective sleeve.

8. The vessel of claim 1, wherein the vessel is IV tubing within a light-protective sleeve.

9. The vessel of any one of claims 1 to 8, wherein the plinabulin is at a concentration of about 40 mg/10 ml in the liquid formulation.

10. The vessel of any one of claims 1 to 9, wherein comprising 60:40 (wt:wt) of propylene glycol and polyoxyl 15 hydroxystearates (PEG15-hydroxystearates).

11. The vessel of any one of claims 1 to 10, wherein the liquid formulation comprises less than 0.5% ether impurity.

12. The vessel of any one of claims 1 to 11, wherein the liquid formulation comprises less than 0.5% alcohol impurity.

13. The vessel of any one of claims 1 to 12, wherein the liquid formulation comprises less than 0.5% water.

14. The vessel of any one of claims 1 to 13, wherein the liquid formulation comprises less than 2.0% total impurities.

15. A kit, comprising:
plinabulin in a light-protective container; and
a light-protective packaging,
wherein, the light-protective container blocks at least 85% of UV/visible light between 290 nm and 450 nm.

16. The kit of claim 15, wherein the plinabulin is at a concentration of about 40 mg/10 ml in a liquid formulation.

17. The kit of claim 15 or 16, wherein the plinabulin is in a liquid formulation comprising propylene glycol and polyoxyl 15 hydroxystearates (PEG15-hydroxystearates).

18. The kit of claim 17, wherein the liquid formulation comprises 60:40 (wt:wt) of propylene glycol and polyoxyl 15 hydroxystearates (PEG15-hydroxystearates).

19. The kit of any one of claims 15 to 18, wherein the light-protective container is an amber vial.

20. The kit of any one of claims 15 to 19, wherein the light-protective container further comprises a rubber stopper and a flip-off cap.

21. The kit of claim 20, wherein the rubber stopper is a chlorobutyl rubber stopper with a B2-40 coating.

22. The kit of claim 20 or 21, wherein the rubber stopper further comprises a plug portion, wherein the plug portion comprises a Flurotec coating.

23. The kit of any one of claims 15 to 22, wherein the light-protective packaging comprises solid black printing on its interior.

24. The kit of any one of claims 15 to 23, further comprising an IV bag.

25. The kit of claim 24, wherein the IV bag is configured to block at least 85% of UV/visible light between 290 nm and 450 nm.

26. The kit of claim 25, wherein the IV bag comprises a light-protective sleeve.

27. The kit of any one of claims 15 to 26, further comprising IV tubing.

28. The kit of claim 27, wherein the IV tubing is configured to block at least 85% of UV/visible light between 290 nm and 450 nm.

29. The kit of claim 28, wherein the IV tubing comprises a light-protective sleeve.

30. A method of administering plinabulin to a subject in need thereof, the method comprising:
providing plinabulin in a light-protected IV bag; and
administering plinabulin to a subject in need thereof through light-protected IV tubing.

31. The method of claim 30, wherein the light-protected IV bag blocks at least 85% of UV/visible light between 290 nm and 450 nm.

32. The method of claim 30 or 31, wherein the light-protected IV tubing blocks at least 85% of UV/visible light between 290 nm and 450 nm.

33. The method of any one of claims 30 to 32, wherein providing the plinabulin comprises injecting the plinabulin into the light-protected IV bag.

34. The method of any one of claims 30 to 32, wherein providing the plinabulin comprises injecting the plinabulin into a non-light-protected IV bag and then placing a light-protecting sleeve over the non-light-protected IV bag.

35. The method of claim 33 or 34, wherein the plinabulin is injected as a liquid formulation into D5W.

36. The method of claim 35, wherein the liquid formulation comprises propylene glycol, and polyoxyl 15 hydroxystearates (PEG15-hydroxystearates).

37. The method of claim 36, wherein the liquid formulation comprises 60:40 (wt:wt) of propylene glycol and polyoxyl 15 hydroxystearates (PEG15-hydroxystearates).

38. The method of any one of claims 35 to 37, wherein the liquid formulation comprises plinabulin is at a concentration of about 40 mg/10 ml.

39. The method of any one of claims 30 to 38, comprising placing non-light-protected IV tubing within a light-protected sleeve to form the light-protected IV tubing.
